(19) United States
(12) Patent Application Publication
(10) Pub. No.: US 2004/0249284 A1
(43) Pub. Date: Dec. 9, 2004

(54) Vector Doppler utilizing constancy of vector flow direction

(75) Inventor: David Vilkomerson, Princeton, NJ (US)

Correspondence Address: Plevy & Howard, 600 North Easton Road, Willow Grove, PA, 19090, US

(73) Assignee: DVX, INC.

(21) Appl. No.: 10/780,241

(22) Filed: Feb. 17, 2004

Related U.S. Application Data
(60) Provisional application No. 60/447,863, filed on Feb. 14, 2003.

(51) Int. Cl. A61B 8/06

(52) U.S. Cl. 600/453

(57) ABSTRACT

A system for improving vector Doppler velocity measurements by using information about the constancy of the velocity's angle to reduce the errors caused by the intrinsic variability in power of Doppler signals.

### RELATED APPLICATIONS

This application claims the benefit of U.S. Provisional Application No. 60/447,863 filed Feb. 14, 2003.

### FIELD OF THE INVENTION

The present invention relates to devices that use multiple beams of ultrasound to determine the velocity of a scattering fluid, and more particularly to Doppler diagnostic medical systems and methods for measuring blood flow.

### BACKGROUND OF INVENTION

Those skilled in the art will appreciate that Doppler ultrasound measurements of velocity, widely used for blood flow measurement in medical applications, and for the measurement of other scattering fluids in industrial applications, depend upon the Doppler effect, whereby a scatterer produces a change in the frequency of the ultrasound that it scatters. This change in frequency is proportional to two unknown quantities: the absolute magnitude of the velocity vector characterizing the motion of the scatterer, and the angle between the velocity vector and the insonating beam.

By simultaneously making two Doppler measurements of a velocity whose vector is coplanar with a transducer using two beams at known angles to each other, the resulting Doppler equations (each of which contains the unknown quantities of absolute value V and angle in the plane [symbol] ) can be solved simultaneously to calculate the velocity and angle to the transducer of that vector.

Determining three vector components of velocity by means of multiple Doppler equations has also been discussed, for example, in U.S. Pat. No. 5,738,097 issued to Beach et al., and U.S. Pat. No. 5,488,953 and U.S. Pat. No. 5,5540,230. These patents teach apparatus and methods useful for pulsed Doppler, rather than continuous wave (CW) Doppler. For certain applications where skilled operators, to interpret the image in order to place the sampling gate needed for pulsed Doppler, are not available (as for primary care screening for disease), CW Doppler is desirable. U.S. Pat. No. 4,062,237, issued to Fox, utilizes crossed CW beams and multiple frequencies where pairs of transducers operate at different frequencies so as to set up a difference frequency standing wave in the region of interest (equivalent to sensitive volume in this disclosure) in order to detect a Doppler frequency.

The method of using multiple Doppler measurements to determine the vector components of the velocity has been used by Daigle (1974 Doctoral Dissertation, Colorado State University) and implemented in previous patents, such as U.S. Pat. No. 5,488,953 entitled, “Doppler Diffracting Transducer” and U.S. Pat. No. 5,540,230 entitled “Doppler Diffracting Transducer”, both issued to Vilkomerson, the inventor herein. These patents, in addition to U.S. Pat. No. 6,346,081 (the '081 patent) entitled “Angle Independent Continuous Wave Doppler Device” disclose means and methods of using special transducers, known as diffraction-grating-transducers (DGTs), to generate the multiple beams needed to effect this method. U.S. Pat. Nos. 5,488,953 and 5,5540,230 teach the use of DGTs for pulsed operation, and the '081 patent, incorporated herein by reference, describes DGTs for CW operation. CW operation is often desirable for medical and some industrial uses because CW operation does not require adjustment of a “sample gate” to define the spatial region in which the Doppler system will measure the velocity. Instead, the region where the beams overlap define a “sensitive region”. The '081 patent describes how this sensitive region is determined for CW operation. The '081 patent also describes a CW, angle-independent system that is orientation independent.

Referring to FIG. 1A, there is shown a vector Doppler transducer system including a transmitting transducer 4 and receiving transducers 1, 2 and 3. The vector Doppler transducer system utilizes three (or more) Doppler measurements arranged so that the measurements involve the three spatial components of velocity, i.e. Vₓ, V_{y}, and V_{z}. Once the three components are determined, the absolute velocity V can be calculated as equal to (Vₓ²+V_{y}²+V_{z}²)^{1/2}. FIG. 1B shows the overlap between the central transmitting beam B_{T}, generated by transmitting transducer 4 and one of the receiving beams B_{R} received by one of the receiving transducers 1, 2, 3. (Only one beam is shown for the sake of clarity).

Using the vector Doppler transducer system of FIGS. 1A and 1B is particularly desirable when measuring blood flow under the skin, where the orientation of the blood vessel is not obvious. With this transducer system, the velocity will be accurately determined independently of the orientation, as was demonstrated experimentally in the article entitled, “Low-Cost Vector Doppler System Utilizing Diffraction-Grating Transducers”, by Vilkomerson et al, Proc. 2000 IEEE International Ultrasonics Symposium, IEEE Press, Piscataway (2001) which is incorporated herein by reference. Details of the vector Doppler transducer system are provided in this article, which describes that three independent Doppler frequency signals containing three corresponding unknown velocity components in three spatial dimensions, i.e. Vₓ, V_{y}, and V_{z}, are analyzed in order to obtain velocity in terms of the three measured Doppler frequencies.

The found spatial velocity vector components Vₓ, V_{y}, and V_{z} define the angle of the velocity vector in space, as shown in FIG. 2, i.e. the Cartesian vector components in x, y, z describe a vector of length V equal to (Vₓ²+V_{y}²+V_{z}²)^{1/2} at an angle θ, equal to the arctangent of V_{y}/Vₓ, and angle φ, equal to the arctangent of V_{z}/(V_{y}/sinθ), i.e.

θ=tan⁻¹(V_{y}/Vₓ) and φ=tan⁻¹[V_{z}/(V_{y}/sinθ)] (1)

The temporal change in the angle of the velocity vector is often much slower than the variation in its length, i.e. the velocity increases and decreases with every beat of the heart, but the direction of flow in space, for example in a blood vessel, will remain constant. Criton et al, in U.S. Pat. No. 6,464,637 used the derived angle to provide for automatically adjusting the “flow” indicator in duplex Doppler diagnostic systems. In recent publications (R. Steel, et al, “Velocity Fluctuation Reduction in Vector Doppler Ultrasound Using a Hybrid Single/Dual-Beam Algorithm”, IEEE Transactions on Ultrasonics, Ferroelectrics and Frequency Control, Vol. 50, page 89, January, 2003, and A. Criton et al, “Real Time Vector Doppler for Tissue Motion”, presented orally in October, 2002, and to be published in Proc. Of 2002 IEEE International Ultrasonics Symposium, IEEE Press, Piscataway, N.J.), it has been shown that by low-pass (temporal) filtering, the calculated angle of flow (a single angle in these publications, as two-beam methods, suitable for use in a plane, are employed), and using this angle to deduce the absolute velocity from the Doppler shift from the optimal (lowest angle-to-flow) beam, the variation in the velocity can be reduced.

While this use of the constancy of the orientation of the velocity vector in space has been shown to be helpful, these published methods do not address a more basic problem in making more reliable Doppler measurements. Doppler measurements are affected by the inherent variability in determining the Doppler frequency caused by the random variation in power around the mean power of Doppler signals.

### SUMMARY OF THE INVENTION

A method is disclosed for improving vector Doppler velocity measurements by using information about the constancy of angle to reduce the effects of the intrinsic power variability Doppler frequency determination.

### BRIEF DESCRIPTION OF THE DRAWINGS

FIGS. 1A and 1B respectively show an end view and a partial side view of an exemplary vector Doppler system, similar to the one used in performing the experiments reported further on in the specification.

FIG. 2 illustrates how the velocity vector in space is described both in terms of the vector components Vx, Vy, and Vz, and by an absolute length, V=(Vx, Vy, Vz)^{1/2}, θ, and φ.

FIG. 3 is a graph showing the average of 150 Doppler power spectra from one receiving transducer measuring a constant-flow of blood-mimicking fluid (solid trace) with one of these spectra (dotted with circles at each bin) superimposed. The velocity of the fluid was 150 cm/sec, and the transducer of FIGS. 1A and 1B was used. In these experiments, each bin represents a band of frequencies of 39 hertz, so for example, bin number 100 represents 3900±19.5 Hz.

FIG. 4A is a graph showing the peak Doppler frequencies f1, f2, f3 versus time from each of the three receiving transducers for the constant flow experiment described in FIG. 3.

FIG. 4B is a graph showing the resulting calculated velocity versus time. Ideally, these would all be flat lines, but the random power levels in the frequency bins for each spectrum, shown in FIG. 3, cause the threshold to be crossed at random points around the “average” threshold point, leading to the “noisy” peak frequency values.

FIGS. 5A and 5B are graphs showing angle θ (FIG. 5A) and angle φ (FIG. 5B) calculated versus time from eqs. (2) and (4) using the peak frequencies shown in FIGS. 4A and 4B. The time-averaged value for these angles is also shown.

FIG. 6 is a flow chart of illustrating an exemplary embodiment of the method of the invention wherein the random variations in f1, f2, and f3 are corrected by using the known true angles θ and φ. The factors relating the frequency terms to the vector components of the velocity are those of Eq. 2 when the values of 42 degrees and 5 MHz, the transducer and frequency of the experimental data, is used. This particular flow chart is applicable for velocities in one direction, as this condition applies to the phantom and in vivo experiments presented.

FIGS. 7A and 7B are graphs presenting the experimental validation of the method of FIG. 6, using the data from the phantom with constant velocity presented in the previous FIGS. 4A, 4B, 5A, and 5B. The corrected velocity is shown as solid, and the uncorrected as dotted; the graph of FIG. 7B is the central portion of the graph of FIG. 7A, expanded so that the improved constancy of velocity is more visible.

FIGS. 8A and 8B are graphs showing velocity measurements of blood flow in a dialysis-access graft; the solid line represents the corrected velocity, and the dotted, the un-corrected. The graph of FIG. 8B is an expanded view of a section of the graph of FIG. 8A, showing how the corrected velocity appears more realistic than the uncorrected velocity.

### DETAILED DESCRIPTION OF THE INVENTION

The present invention is a method that reduces the variability in the velocity calculated by vector Doppler when used where the orientation of the velocity vector is constant in space, e.g. the blood velocity measured in an artery. The method of the invention may be utilized in any suitably arranged Doppler ultrasound system which employs an ultrasound transducer configuration that uses two or more beams. The method may implemented in such a system as software, hardware, or as a combination of software and hardware. For example, the method may be implemented as computer-executable instructions and data stored on a hard disk drive of a personal computer or on a removable storage medium which may be a CD-ROM disk, a DVD disk, a floppy disk or the like.

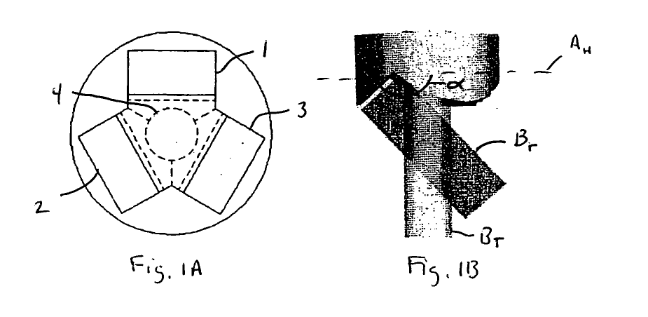

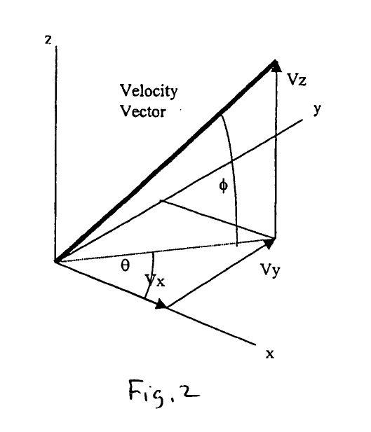

In the earlier described vector Doppler transducer systems, as exemplified in FIGS. 1A and 1B, the vector velocities are calculated from the two (or more) Doppler shift frequencies measured. These frequencies are calculated from the spectrum of the Doppler frequencies, usually derived by an FFT procedure, as is described in detail in D. Evans and W. McDicken Doppler Ultrasound, Chapter 8, Spectral Estimation Techniques, Wiley, Chichester, 2000. The maximum velocity is required for several important medical diagnostic indicators, in particular determining the degree of stenosis of a vessel. To determine the peak velocity, the peak frequencies in the beams must be determined.

Because the Doppler signal is the result of the sum of the ultrasound signals scattered by the individual red blood cells in blood, and these scatterers are randomly distributed, the Doppler signal amplitude, while on the average proportional to the number of scatterers, varies significantly around its average value. As is well-known to those skilled in the art, the power of such a signal is described as a Rayleigh distribution (corresponding to the statistical function of chi-squared distribution with degree of freedom 2), with the average power proportional to the number of scatterers, and the variance of the power equal to a constant percentage of the mean power.

This variation in power is also true for the power in a given frequency band of the Fourier Transform power spectrum of the Doppler signal. To find the peak frequency (which leads to the peak velocity desired), typically a threshold is established, e.g. .9 of the average peak power in the spectrum: the highest frequency bin whose power exceeds the threshold is determined to be the peak frequency (D. Evans and W. McDicken, Doppler Ultrasound, Chapter 8, op. cit.).

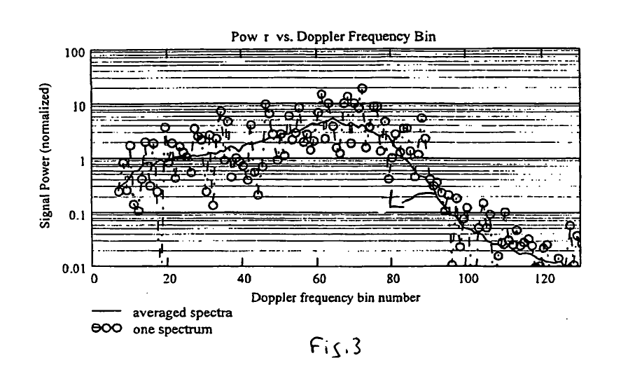

However, because the power in any range of frequencies in the Doppler spectrum is described by the Rayleigh distribution, the powers observed in each “bin” will vary markedly from spectrum to spectrum, even if the true velocity is constant. For example, FIG. 3 shows the Doppler signal-power spectra measured from blood-mimicking fluid flowing in a tube in laminar flow at a constant rate. Each Doppler spectrum presented resulted from taking the absolute magnitude squared of the Fourier Transform of 512 consecutive samples, at 20 KHz sampling rate, of the Doppler shift in frequency. When 150 Doppler spectra are averaged together, averaging out the variation due to the Rayleigh distribution of signal strength, solid line L is obtained, as expected showing a distribution of fluid velocities, from low velocity, near the wall, to the peak velocity. The “peak frequency” of these averaged spectra is easy to determine. However, if a single spectrum of that set of spectra is examined, shown as circles in FIG. 3, the large variations between the power in each frequency bin (a band of frequencies, in the case shown, of 39 Hz) would make determining the peak frequency uncertain. While on the average the threshold will provide the maximum frequency, for any one spectrum the peak frequency will have a variable component, as sometimes the bin whose average power is below the threshold will exceed the threshold, leading to using an erroneous high peak frequency, and sometimes the bins that should meet or exceed the threshold will be below the threshold, leading to using an erroneous low peak frequency.

(It should be noted that each Doppler frequency component can only be calculated to a degree of accuracy determined by its observation time, e.g. if the blood cells cross the sensitive volume in ten milliseconds, we can determine its Doppler frequency only to ±100 cycles. This is known to those skilled in the art as intrinsic spectral broadening (Doppler Ultrasound, op. cit, page 134) and produces the slope at the end of the averaged spectrum in FIG. 3.)

Thus, the peak frequencies determined will always have an intrinsic variability due to the random power caused by the random positions and orientations of the scatterers. This variation will produce variability in the velocity calculated from the peak frequencies in vector Doppler, even if the vector angle, by being low-passed filtered as suggested in the recent referenced publications, is stable.

The embodiments of the method of the present invention described below are directed at reducing the variability in the calculation of the peak frequencies due to the random nature of the Doppler power, and thus increasing the reliability of vector Doppler measurements of blood velocity. In general terms, the method of the invention uses the constancy of the velocity vector spatial orientation to correct for the errors due to the random nature of the Doppler signal, rather than temporally filtering the vector orientation as in existing systems. Here, the use of the extra information known about the signal, i.e., the constancy of the velocity vector orientation, enables error-correction, in the same way as extra bits are used in error-correcting codes used in communication.

Another condition for the method of the present invention to be used is that the velocity measurement must not be required to be instantaneous. As will be shown, the method of the invention depends upon taking several hundred spectra before the (corrected) velocity in these spectra is displayed. This condition makes this method particularly suitable for monitoring applications, where instantaneous velocity measurement is not required, or in screening situations, where a measurement is taken and the results tabulated at a later time.

Another advantage of the method of the present invention is that it can detect errors in the calculated velocities.

The method of the invention utilizes the angle information inherent in the vector Doppler determination to correct for the errors due to the random power in the signal. The velocity vector V, whose length is proportional to the velocity, has vector components Vx, Vy, Vz in Cartesian coordinates that define the angle of the vector in space, i.e. angles θ and φ as shown in FIG. 2. When the flow whose velocity is to be measured is constrained to a particular orientation in space, for example, when it describes the flow of blood through an artery or dialysis-access tube, the length of the velocity vector, V, will vary with time but the velocity vector's orientation in space, described by angles θ and φ, will not. We can use this knowledge to find the true angles θ and φ by averaging the angle θ and φ components over time, e.g. a hundred or more spectra over several heart cycles, to average-out their variability. We then use the knowledge of the true orientation in space to enable us to calculate “correction factors” for each peak frequency—the correction factors can be found by iterating around the threshold-detected frequencies (which include the errors due to the Rayleigh power distribution as noted above) until the sum of the correction factors and the threshold-detected frequencies produce the true (as found by the averaging) vector angle. The corrected frequencies, i.e. the sum of the threshold-detected frequencies and their correction factors, are then used to calculate a more reliable velocity.

For clarity, the method of the present invention will be described in terms of a specific, exemplary embodiment. However, it should be understood that the general applications of the invention as discussed above are not limited to any one embodiment.

Referring again to the vector Doppler transducer system of FIGS 1A and 1B, the three receiving transducers 1, 2, 3, are disposed at 120 degree intervals around the central transmitting transducer 4 (FIG. 1A), which may be 14 mm is diameter. The receiving transducers 1, 2, 3 are set at an angle α (e.g. 42 degrees) to the horizontal axis A_{H}, so that where the transmitting beam B_{T} and the receiving beam overlap (FIG. 1B), there is a “sensitive volume” where Doppler measurements can be made. As described in detail in the earlier mentioned article entitled, “Low-cost Vector Doppler System Utilizing Diffraction-grating Transducers”, by Vilkomerson et al., the Doppler frequencies, i.e. the frequency shifted signals generated by the Doppler effect, for the peak frequencies f1, f2, f3 from the three transducers marked as shown in FIG. 2 (with transducer 1 oriented on the y-axis and the transmitted beam on the z-axis) are:

f1=(1/λ){V_{y}sin α+V_{z}(1+cos α)}

f2=(1/λ)[−Vₓcos 30°sin α−V_{y}sin 30°sin α+V_{z}(1+cos α)]

f3=(1/λ)[Vₓcos 30°sin α−V_{y}sin 30°sin α+V_{z}(1+cos α)

When these equations are simultaneously solved for the three vector components Vx, Vy, Vz in terms of the measured peak frequencies f1, f2, f3, it is found that:

Vₓ=λ(f3−f2)/√3 sin α

V_{y}=λ(2f1−(f2+f3)/3 sin α (2)

V_{z}=λ(f1+f2+f3)/3(1+cos α)

From the three vector components Vₓ, V_{y}, V_{z}, the absolute velocity can be calculated as the length of the velocity vector:

V=(Vₓ²+V_{y}²+V_{z}²)^{1/2} (3)

The vector components for calculating the peak velocity are derived from the peak frequencies, f1, f2, and f3, as described by equation 2 above. The peak frequencies will vary, even if the velocity of the blood does not, because of the random power variation (Rayleigh distribution) shown in FIG. 2.

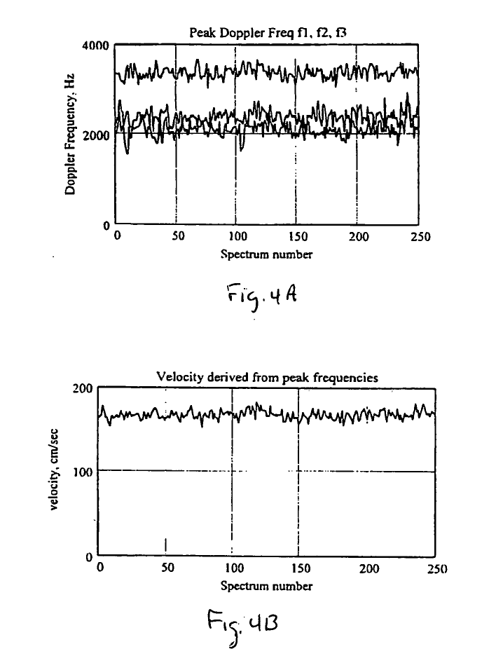

For example, FIGS. 4A and 4B show the peak frequencies, calculated with a set threshold as described earlier, obtained with constant flow of blood-mimicking fluid, over the course of 3 seconds (250 spectra). Ideally, the peak frequencies would be constant, leading to a constant peak frequency. However, as expected for Rayleigh distributed power in the signals, each frequency shows fluctuations, leading to the overall fluctuating velocity shown in FIG. 4B. (The velocity deviation is less than each frequency deviation because it represents the summation of 3 independent random components.)

In FIG. 2, the velocity vector of absolute value V described by Vx, Vy, Vz is shown in space, along with the components of angles θ, and φ. Observing FIG. 2, it can be seen that Vp, the projection of V onto the x-y plane in the figure is of magnitude

Vp=(Vx²+Vy²)^{1/2} and

φ=a cos(Vp/V) and θ=a sin(Vp/Vx) (4)

allowing angles θ and φ to be derived from the vector components Vx, Vy, and Vz calculated in Eq. 2 above.

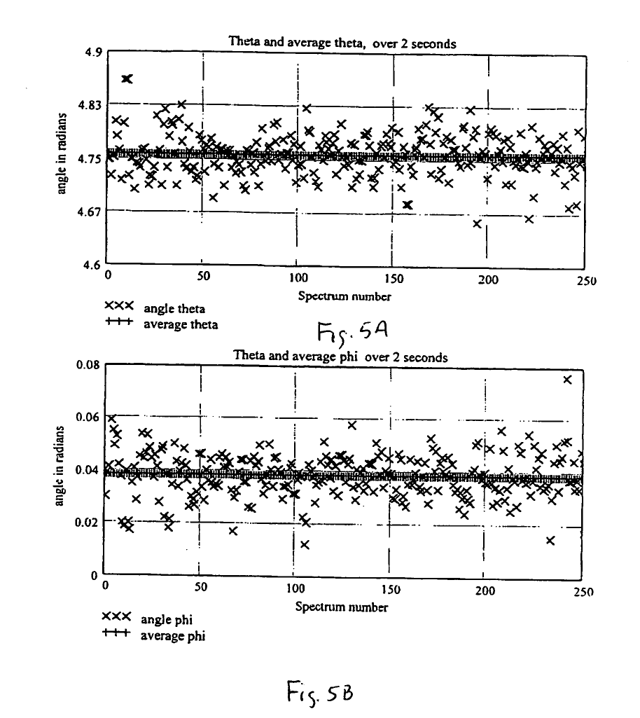

In FIGS. 5A and 5B, the angles θ and φ of the velocity vector V, derived from the frequencies shown in FIG. 4, are plotted vs. time, along with their average values over the 3 seconds of flow measured. The variability in the peak frequencies f₁, f₂, f₃ shown in FIG. 4A leads to variation in the angles θ and φ apparent in FIGS. 5A and 5B. However, as the tube through which the blood-mimicking fluid is flowing is not moving, we know that in reality these angles must be constant. It is this “extra information” that allows correction of the random errors in the velocity.

The method of the present invention, as described below with reference to FIG. 6, corrects the peak frequencies so that a better measure of V can be obtained.

In step 30, it is assumed that each peak frequency determined by the threshold method includes a true peak frequency, ft, plus an error frequency fe (which can be either positive or negative):

f1=f1t+f1e; f2=f2t+f2e; f3=f3t+f3e;

In step 20, the “true” angles θ and φ of the velocity vector are determined by averaging a sum of measurements of angles θ and φ over time, recognizing that averaging out the random variations over many measurements reduces the random components with relation to the true value. The number of spectra that should be averaged is determined by the degree of variability that is to be achieved, and may depend on the particular situation of measurement.

In steps 40, 50, 60, the probable error frequencies fe in each frequency is calculated by finding the three error frequencies, f1e, f2e, and f3e, that minimize the difference between the angle calculated for each measurement and the “true” angles calculated in step 20.

In step 70, corrected frequencies, i.e. with the error frequencies subtracted from the measured frequencies, e.g. f1t=f1−f1e, etc., are used to calculate the correct velocity, according to the equation derived in the paper, “Low-Cost Vector Doppler System Utilizing the Diffraction-Grating Transducers” previously referenced, and using the wavelength and angle parameters applicable to the Figures herein.

There are a number of different iterative algorithms known to those skilled in the art that may be used for minimizing the error frequencies. The MINERR algorithm shown in step 40 of FIG. 6 is preferred. The MINERR algorithm of Mathcad (Mathcad 11, Mathsoft, Inc.) uses a conjugate gradient method, as described in G. Golub and C. Van Loan, Matrix Computations, 2^{nd} Ed, Johns Hopkins University Press, Baltimore, 1989. A complete algorithm would be optimized for the angles calculated, i.e. use of cotangent rather than tangent around 90°, where tan θ approaches infinity, as will be recognized by those skilled in the art.

Note that there is a special “error trapping” branch that is invoked if the correction term, which starts at fe of zero for each component, becomes larger than is reasonable for the particular parameters of the measurement being made. In this way, errors due to external noise, as opposed to the natural Rayleigh variations in the measurements, can be detected and eliminated.

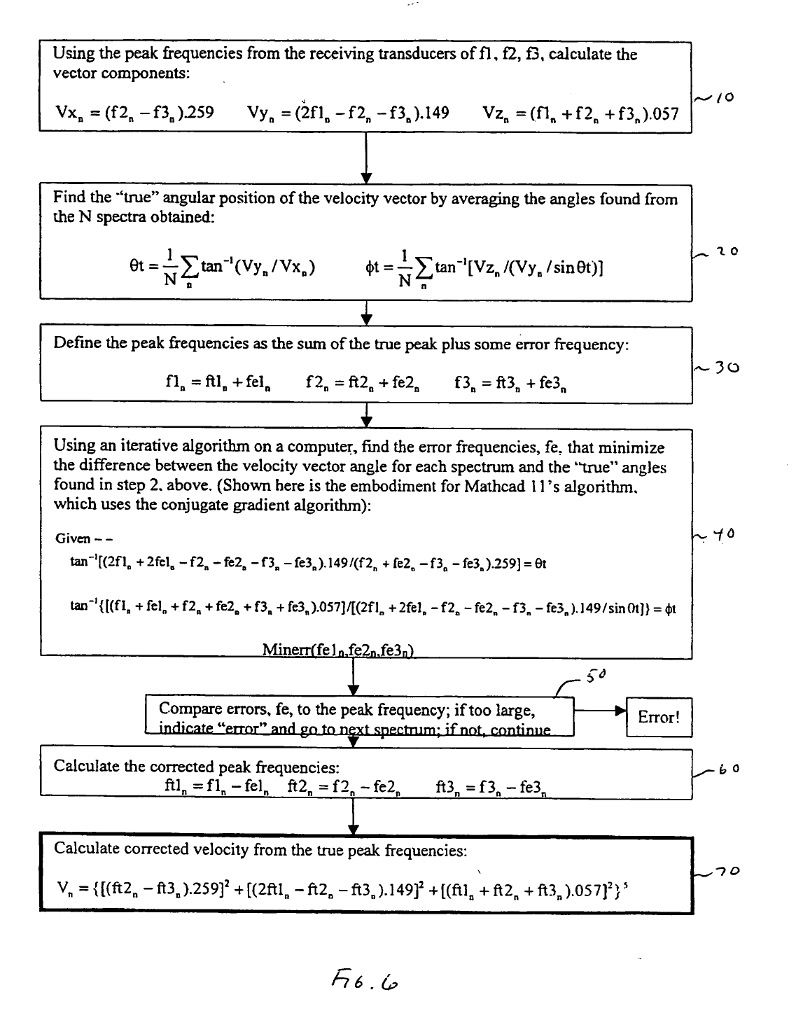

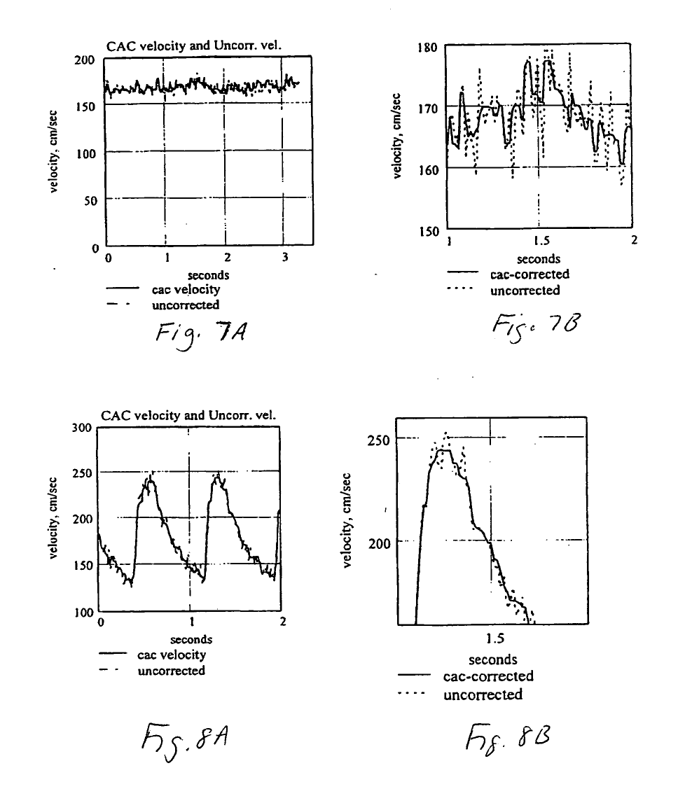

The results of this correction for the flow measurements shown in FIGS. 4A, 4B, 5A and 5B are shown in FIGS. 7A and 7B. FIGS. 7A and 7B show the corrected and uncorrected velocities for the flow data shown in the earlier FIGS. FIG. 7B shows the expanded view of the velocity shown in FIG. 7A. As can be seen, the maximum deviation in velocity has been reduced from 19% to 11%. The solid line is the corrected velocity, as obtained by the procedure shown in the flow chart of FIG. 6, while the dotted line is the original velocity as shown in FIG. 4B. The correction method of the present invention has reduced the range of velocity measurements taken over 3 seconds from ±9.5% to ±5.5%.

The method does not guarantee perfect removal of the variability caused by the random errors because the solution for three unknowns using two equations is not unique; if the errors are approximately the same size, the iteration will find the error terms that remove the random variation. However, sometimes the iterative solution will not be the right one, so the variability in velocity is reduced, rather than eliminated.

FIGS. 8A and 8B show the same correction process applied in vivo on a velocity measurement of blood flow in a dialysis-access graft. More specifically, FIGS. 8A and 8B show the constant-angle-corrected (cac) velocity and uncorrected velocity measured by the prototype instrument on blood flow in a dialysis access graft. FIG. 8A shows a 2 second recording, and the FIG. 8B shows an expanded view of the uncorrected (dotted trace) and corrected (solid trace) velocity calculated, showing the effect of the constant-angle-correction method. The correction process can be used because, while the velocity is variable in time (with the beating of the heart), the transducer system is held steadily over the graft during this measurement, so the orientation of the velocity vector does not change. The corrected velocity, shown as the solid trace in FIG. 8B, is more consistent with hemodynamic fluid flow and so is believed to be closer to the true velocity.

It should now be apparent that the method of the present invention improves vector Doppler velocity measurements by using information about the constancy of its angle to reduce the effects of the intrinsic power variability of Doppler signals. The method of the present invention may also be applied in a straightforward manner to instrument configurations that use more or fewer beams. While the algorithms described here are for mono-phasic flow, i.e. flow in only one direction, those skilled in the art will appreciate that the method of the present invention may be easily adapted to bi-phasic and tri-phasic flows. If additional information about the flow to be measured is available, e.g. the most rapid change in velocity that can be expected, additional criteria reflecting that information can be included in the iterative process, i.e. adding to step 40 in FIG. 6 an additional equation:

Vₙ(corrected)−Vₙ₋₁(corrected)<allowed change in velocity/time.

Another value of the present method is that the process of compensating for the random variations provides for detecting measurement errors larger than those due to the random power intrinsic to Doppler signals.

Although the method of the present invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A method for measuring fluid velocity using Doppler flow measurements from an ultrasound device, the method comprising the steps of:
obtaining information about angle constancy; and
reducing the effects of intrinsic power variability of Doppler frequency determination by using the information about angle constancy.

2. A method for measuring fluid velocity comprising the steps of:
obtaining a plurality of Doppler power spectra from an ultrasound device measuring a fluid velocity, each of the Doppler velocity spectra defined by at least two peak Doppler frequencies;
calculating angular positions of a velocity vector characterizing motion of the fluid velocity, each angular position calculated from the at least two peak Doppler frequencies of each of the Doppler spectra; and
determining a true angular position of the velocity vector from the calculated angular positions.

3. The method according to claim 2, further comprising the step of determining peak Doppler frequency errors.

4. The method according to claim 3, wherein each of the peak Doppler frequency errors is determined by minimizing a difference between a corresponding one of the calculated angular positions of the velocity vector and the determined true angular position of the velocity vector.

5. The method according to claim 2, further comprising the step of determining true peak Doppler frequencies from the peak Doppler frequency errors and the peak Doppler frequencies corresponding therewith.

6. The method according to claim 5, further comprising the step of determining a corrected velocity vector from the true peak Doppler frequencies.

7. The method according to claim 2, wherein the Doppler power spectra obtaining step is performed over a given time period.

8. The method according to claim 7, wherein the true angular position determining step is performed by averaging a sum of the calculated angular positions.

9. The method according to claim 5, wherein the true peak Doppler frequencies are determined by subtracting the peak Doppler frequency errors from corresponding ones of the peak Doppler frequencies.

10. The method according to claim 2, wherein the fluid comprises an ultrasound scattering fluid.

11. The method according to claim 2, wherein the fluid comprises blood.

12. A method for reducing variability in vector Doppler velocity measurements, the method comprising the steps of:
obtaining a plurality of Doppler power spectra from an ultrasound device measuring a fluid velocity, each of the Doppler flow spectra defined by at least two peak Doppler frequencies;
calculating angular positions of a velocity vector characterizing motion of the fluid flow, each angular position calculated from the at least two peak Doppler frequencies of each of the Doppler velocity spectra; and
determining a true angular position of the velocity vector from the calculated angular positions.

13. The method according to claim 12, further comprising the step of determining peak Doppler frequency errors.

14. The method according to claim 13, wherein each of the peak Doppler frequency errors is determined by minimizing a difference between a corresponding one of the calculated angular positions of the velocity vector and the determined true angular position of the velocity vector.

15. The method according to claim 12, further comprising the step of determining true peak Doppler frequencies from the peak Doppler frequency errors and the peak Doppler frequencies corresponding therewith.

16. The method according to claim 15, further comprising the step of determining a corrected velocity vector from the true peak Doppler frequencies.

17. The method according to claim 12, wherein the Doppler power spectra obtaining step is performed over a given time period.

18. The method according to claim 17, wherein the true angular position determining step is performed by averaging a sum of the calculated angular positions.

19. The method according to claim 5, wherein the true peak Doppler frequencies are determined by subtracting the peak Doppler frequency errors from corresponding ones of the peak Doppler frequencies.

20. The method according to claim 12, wherein the fluid comprises an ultrasound scattering fluid.

21. The method according to claim 12, wherein the fluid comprises blood.

22. An ultrasound sound system for use in measuring fluid velocity comprising:
an ultrasound device for measuring a Doppler velocity signal; and
means for using constancy of a velocity vector spatial orientation from the measurement to correct for errors due to a random power characteristic of the Doppler signal.

23. An ultrasound sound system for use in measuring fluid velocity comprising:
an ultrasound device for measuring a Doppler velocity signal; and
means for reducing effects of intrinsic power variability Doppler frequency determination in the measured signal by using information about angle constancy.

24. The device of claim 23, wherein the ultrasound device uses at least two beams.

25. An ultrasound system for measuring fluid velocity, the system comprising:
an ultrasound device for measuring a fluid velocity and obtaining a plurality of Doppler power spectra, each of the Doppler spectra defined by at least two peak Doppler frequencies;
means for calculating angular positions of a velocity vector characterizing motion of the fluid flow, each angular position calculated from the at least two peak Doppler frequencies of each of the Doppler flow spectra; and
means for determining a true angular position of the velocity vector from the calculated angular positions.

26. The ultrasound system according to claim 25, further comprising means for determining peak Doppler frequency errors.

27. The ultrasound system according to claim 26, wherein each of the peak Doppler frequency errors is determined by minimizing a difference between a corresponding one of the calculated angular positions of the velocity vector and the determined true angular position of the velocity vector.

28. The ultrasound system according to claim 25, further comprising means for determining true peak Doppler frequencies from the peak Doppler frequency errors and the peak Doppler frequencies corresponding therewith.

29. The ultrasound system according to claim 28, further comprising means for determining a corrected velocity vector from the true peak Doppler frequencies.

30. The ultrasound system according to claim 25, wherein the Doppler power spectra is obtained over a given time period.

31. The ultrasound system according to claim 30, wherein the true angular position is determined by averaging a sum of the calculated angular positions.

32. The ultrasound system according to claim 28, wherein the true peak Doppler frequencies are determined by subtracting the peak Doppler frequency errors from corresponding ones of the peak Doppler frequencies.

33. The ultrasound system according to claim 25, wherein the fluid comprises an ultrasound scattering fluid.

34. The ultrasound system according to claim 25, wherein the fluid comprises blood.
